(19) United States
(12) Patent Application Publication
(10) Pub. No.: US 2013/0110147 A1
(43) Pub. Date: May 2, 2013

(54) TUBE SET FOR A ROTARY TISSUE CUTTER WITH CURVED INNER BLADE

(71) Applicant: Randy Dame, High Ridge, MO (US)

(72) Inventor: Randy Dame, High Ridge, MO (US)

(21) Appl. No.: 13/285,390

(22) Filed: Oct. 31, 2011

(51) Int. Cl. A61B 17/32

(52) U.S. Cl. 606/180

(57) ABSTRACT

Interacting cutting blades, forming a part of a rotary tissue cutter, has an outer cutting blade 28 including an edge 36 defining a port 34 formed near a distal end 30 of the outer cutting blade 28. An inner cutting blade 12 for rotation relative to the outer cutting blade 28 is held within the outer cutting blade 28. The inner cutting blade 12 includes one or more cutting elements 20 formed near a distal end 18 such that an edge 14 of the inner cutting blade 12 forms resilient frictional contact with the edge 36 and the cutting elements 20 each have a convex shape such that an apex 22 of each of the cutting elements 20 extends within the port 34 and beyond an inner surface 38 of the outer cutting blade 28 as the inner cutting blade 12 is rotated within the outer cutting blade 28.

### FIELD

The present disclosure relates to a rotary tissue cutter with an inner blade having a portion that is curved. More specifically, the present disclosure relates to a tube set having inner and outer interacting blades where the inner blade is curved so that a portion of the inner blade is received within a port of an outer blade, to create a scissors cut.

### BACKGROUND

This section provides background information related to the present disclosure that is not necessarily prior art.

Tissue cutters, including vitreous cutters, are well known surgical instruments used in vitreoretinal or posterior segment surgery to dissect vitreous and aspirate the dissected vitreous from the eye, usually in preparation for other surgical procedures. Vitreous cutters can be driven pneumatically, electrically, or otherwise.

Most vitreous (vit) cutters include a pair of cutting blades formed as tubes with an inner blade held within an outer blade. The inner blade typically has some blade-shape formed near a distal end that cooperates with a port formed near a distal end to cut tissue aspirated into the port via a suction pump connected through the tubes. The inner blade typically is made to move across the port to cut by a combination of a motor force and mechanical linkage between the motor force and the inner and/or outer blade.

The most common type of vit cutter is a linearly reciprocating cutter having the inner blade move back and forth across the port along the longitudinal axes of the blades. Some have referred to reciprocating vit cutters as guillotine cutters. Reciprocating cutters have been found to reliably cut tissue without danger of traction from the vitreous. Traction occurs when elastic strands of vitreous are not completely severed by the vit cutter as vitreous continues to be aspirated potentially causing complications, such as detaching the retina if the un-severed but aspirated vitreous is attached to the retina.

Another known type of vit cutter is a rotating cutter having the inner blade spinning across the port or reciprocating back and forth across the port orthogonally to the longitudinal axes of the blades. Historically, rotating vit cutters have experienced greater traction issues compared to reciprocating vit cutters. It is believed that rotating vit cutters do create sufficient frictional contact between the inner blade and the port to ensure a clean scissors-like cut. Known linearly reciprocating vit cutters disclose many ways to ensure a proper scissor cut, and typically include altering the inner blade in a manner that ensures frictional contact along the entire edge of the port as the inner blade moves across the port. Frictional contact is typically accomplished by bending the inner or outer blade relative to the other blade, creating a slit and flaring the inner blade at the distal end, removing a portion of the inner blade distal end and tapering the outer blade distal end, creating a ramp or indentation in the outer blade that forces the inner blade toward the port, etc. Rotating blades also use techniques, such as creating a flared distal portion to attempt to create frictional contact. FIG. 1 is an example of a prior art inner blade 2 having straight edges 4 at a distal end of blade 2. Notches 6 are formed during manufacture allowing the blade 2, including edges 4, to be flared outwardly to increase the diameter of blade 2 in the portion, including edges 4 relative to the other portions of blade 2. This flaring is to provide frictional contact with an outer blade 8 at a location corresponding to a port 10, as seen in FIG. 2. It is believed that the straight edges 4 do not make sufficient frictional contact across the entire length port 10 to ensure all vitreous is severed as it is being aspirated into port 10.

Rotating vit cutters are potentially desirable for a number of reasons, including because they can use a greater variety of motor forces with less complicated linkages between the motor force and the inner blade compared to the linearly reciprocating cutters. For example, a rotating vit cutter could employ an electric motor with a drive shaft directly attached to the inner blade, eliminating the need for a transmission converting the rotation of the electric motor to linear reciprocating motion required to a linearly reciprocation vit cutter.

It is believed that traction issues arise with rotating vit cutters, because even with a flared distal end, the inner blade does not make sufficient frictional contact with the entire periphery of the port, allowing some vitreous to not be severed.

Contrary to the above examples, the outer blade could be caused to move relative to a stationary inner blade or both blades could be moved relative to each other to sever tissue.

Hence, providing a rotating vit cutter that ensures a scissors-like cut of vitreous that is equally or more reliable than known linearly reciprocating vit cutters would be desirable.

### DRAWINGS

The drawings described herein are for illustrative purposes only of selected embodiments and not all possible implementations, and are not intended to limit the scope of the present disclosure.

FIG. 1 is a perspective view of a portion of a prior art rotating vit cutter inner blade;

FIG. 2 is a partial cut-away view of the inner blade of FIG. 1 inserted into an outer blade;

FIG. 3 is a perspective view of a portion or an inner blade corresponding to an example of the present disclosure; and

FIG. 4 is a perspective view of a portion of a rotating vit cutter corresponding to an example of the present disclosure.

Corresponding reference numerals indicate corresponding parts throughout the several views of the drawings.

### DETAILED DESCRIPTION

Example embodiments will now be described more fully with reference to the accompanying drawings.

Example embodiments are provided so that this disclosure will be thorough, and will fully convey the scope to those who are skilled in the art. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

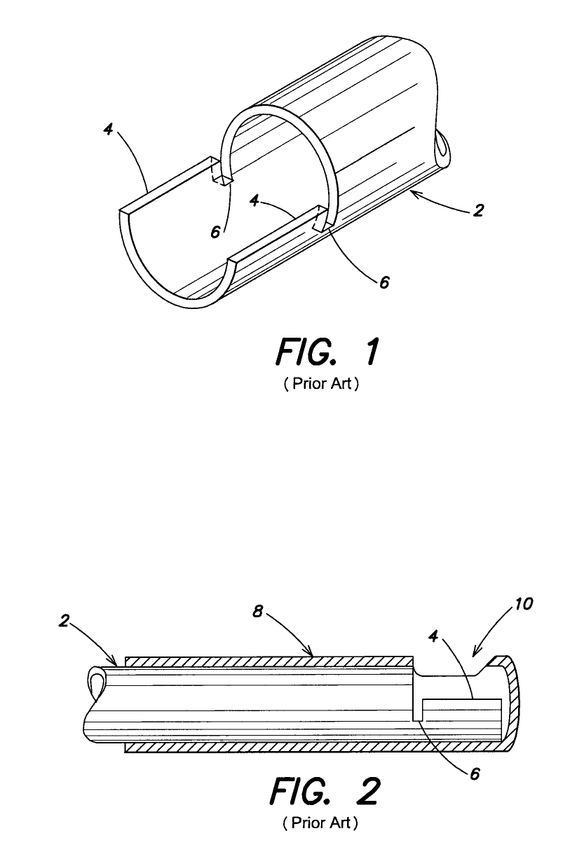

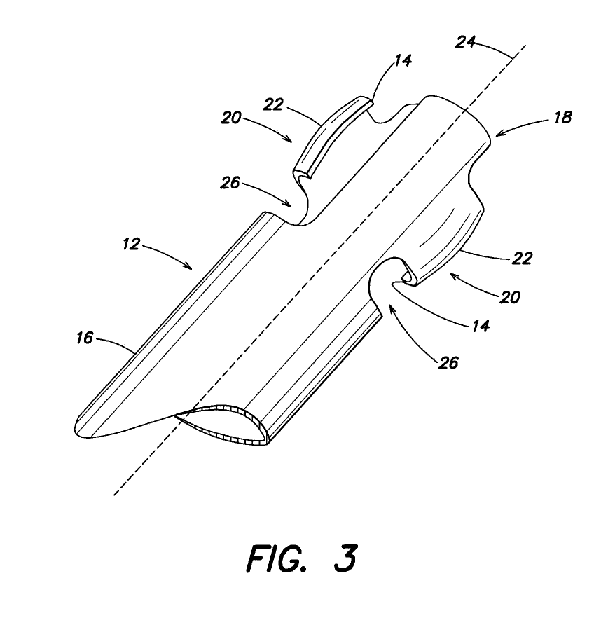

FIG. 3 shows a portion of an inner cutting blade 12 for a rotary or rotating vit cutter that ensures sufficient frictional contact between cutting edges 14 and an edge of a port of an outer cutting blade, shown below. Inner blade 12 is has a generally tubular shaft portion 16 that is connected to a cutter housing and drive mechanism at its proximal end (not shown). A distal end 18 of inner blade 12 has cutting elements 20 formed from the shaft 16, including cutting edges 14. As seen, cutting elements are curved in multiple directions and can be described as convex is shape with an apex, shown generally at reference 22. Cutting elements 20's convex shape differs from prior art inner rotary blades, such as shown in FIG. 1, that have cutting elements that are curved only with respect to the longitudinal axis of the tubular shaft to allow rotation within an outer cutter, but are straight otherwise. For example, prior art edges 4 are straight and not curved like cutting edges 14 of the current disclosure. Notches 26 may be formed in shaft 16 to allow cutting elements 20 to be flared relative to shaft 16 to ensure that the cutting edges 14 maintain a constant resilient spring-like frictional contact with the entire edge defining the port in an outer cutter. The flaring can be accomplished by any known technique, such as with the use of a jig. The convex curvature of cutting elements 20 may also be formed by any known technique, such as the use of a jig and a press that causes the material of inner blade 12 to conform to a profile shape of the jig. Flaring results in apex 22 lying in a plane above the outer periphery of shaft 16, as seen in FIG. 3, That is to say the distance between longitudinal axis 24 and apex 22 is greater than the distance between axis 24 and the outer edge of shaft 16. FIG. 3 shows two cutting elements 20, but more or only one cutting element 20 may be formed, depending on the needs and desires of the user and the type of surgery to be performed.

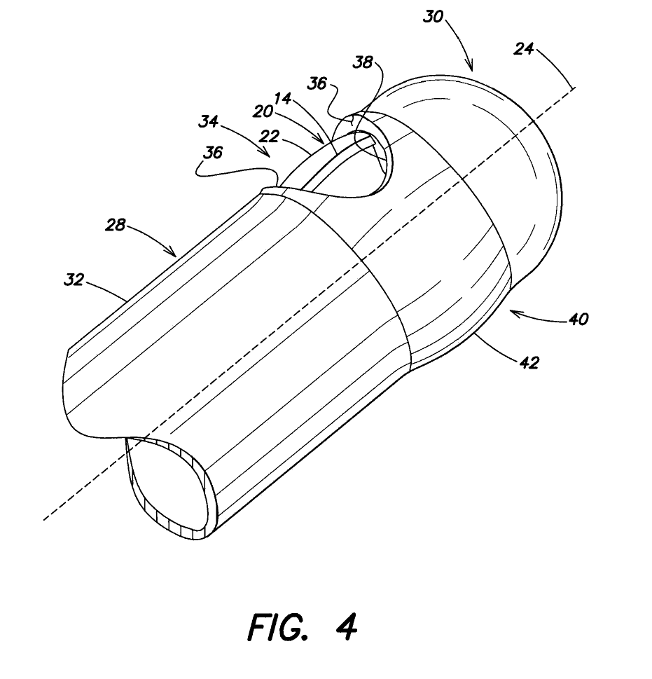

FIG. 4 shows a tube set of the present disclosure with the inner cutting blade 12 of FIG. 3 held within an outer cutting blade 28. Outer blade 28 has a distal end 30 that may be closed by any known technique, such as welding, swaging, crimping, etc. Outer blade 28 has a generally tubular shaft portion 32 connected to a device housing at a proximal end (not shown). The shaft 32 has a port 34 formed near or adjacent distal end 30. Port 34 has a periphery defined by edge 36. The shape of cutting elements 20 allow the cutting edges 14 to make and maintain intimate spring-biased scissors-like contact with the edge 36 to ensure complete safe severing of tissue, especially vitreous tissue during surgery. The shape of cutting elements 20 causes the apex 22 to be located within the port 36 and beyond an inner surface 38 of outer blade 28 so that the necessary frictional contact between edges 14 and 36 is ensured, as the inner cutting blade is rotated within the outer cutting blade. Such intimate contact cannot be ensured by prior art devices with straight cutting elements.

Additionally, outer blade 28 may have a mating curved portion 38 formed at a location corresponding to port 34 so that cutting elements 20 can be easily located or positioned within outer blades 28. Generally, apex 40 of curved portion 38 will correspond to apex 22 of cutting elements 20 relative to a cross-section orthogonal to longitudinal axis 24. Because cutting elements 20 are flared so that the entire span of each edge 14 has a diameter greater than an diameter of inner surface 38, the cutting elements 20 will form a resilient detent-like connection with curved portion 38, thus ensuring easy and quick location of inner cutting blade 12 within outer cutting blade 28. This easy location makes manufacture of vitreous cutters in accord with the present disclosure less expensive, more reliable, and faster compared to prior art vitreous cutters requiring precise fixtures and jigs and labor intensive alignment between inner and outer blades. The curved portion 38 may be formed with the use of a jig and a press to conform the shaft 32 to the shape of the jig.

Curved portion 38 does not necessarily need to be used with the present disclosure, but may provide the advantages mentioned above. For example, during manufacture, and inner blade 12 can be inserted into an outer blade 28 until an assembler feels or detects the cutting elements 20 expanding or springing into curved portion 38. At this point, the assembler reliably knows the cutting elements are aligned properly with port 34 and minimal, if any, further measurements are necessary.

The maximum number of cuts per minute is only limited by the speed of the drive motor/mechanism and the number of cutting elements formed. During use inner cutting blade 12 is rotated within outer cutting blade 28, as negative aspiration pressure is applied to port 34 (via a pump not shown) to pull tissue such as vitreous into port 34. Any tissue pulled into port 34 is severed by rotating inner cutter blade 12 and aspirated away from the surgical site through blades 12 and 28 and collected in a waste container (not shown).

Inner and outer cuffing blades 12 and 28 may be formed of any suitable materials, such as metal, pliable composites, resins, or other such materials that may be formed to sever tissue. The inner and outer cutting blades need not necessarily be formed of the same materials.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof.

Spatially relative terms, such as “inner,” “outer,” “beneath,” “below,” “lower,” “above,” “upper,” “distal,” “proximal,” and the like, may be used herein for ease of description to describe one element or feature's relationship to another elements) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

What is claimed is:

1. A tube set of interacting cutting blades forming a part of a rotary tissue cutter comprising:
an outer cutting blade including an edge defining a port formed near a distal end of the outer cutting blade;
an inner cutting blade for rotation relative to the outer cutting blade is held within the outer cutting blade; and
the inner cutting blade includes one or more cutting elements formed near a distal end of the inner cutting blade such that an edge of the inner cutting blade forms resilient frictional contact with the edge defining the port of the outer cutting blade and the cutting elements each have a convex shape such that an apex of each of the cutting elements extends within the port and beyond an inner surface of outer cutting blade as the inner cutting blade is rotated within the outer cutting blade.

2. The tube set of claim 1, wherein cutting elements are flared relative to a shaft portion of the inner cutting blade to ensure that the edge of the inner cutting blade maintains the resilient frictional contact with the entire edge defining the port.

3. The tube set of claim 1, wherein two cutting elements are formed on a distal end of the inner cutting blade.

4. The tube set of claim 1, wherein a mating curved portion is formed in the outer cutting blade, at a location corresponding to the port, so that the cutting elements can be easily position within the outer cutting blade, such that an apex of the curved portion corresponds to the apex of cutting elements forming a resilient detent-like connection.
